(19) United States
(12) Patent Application Publication
(10) Pub. No.: US 2009/0156969 A1
(43) Pub. Date: Jun. 18, 2009

(54) Method and equipment to induce osteogenesis in a bone region of a patient

(76) Inventor: Massimo Santangelo, Vipiteno (IT)

Correspondence Address: Themis Law, 7660 Fay Ave Ste H535, La Jolla, CA, 92037, US

(21) Appl. No.: 12/281,325

(22) PCT Filed: Mar. 26, 2007
(86) PCT No.: PCT/EP2007/052881

§ 371 Date: Nov. 5, 2008

(30) Foreign Application Priority Data
Mar. 30, 2006 (IT) BO2006A000221

(51) Int. Cl. A61N 7/00; A61B 5/055

(52) U.S. Cl. 601/2; 600/411

(57) ABSTRACT

Apparatus and method to induce osteogenesis in a bone region of a patient, by which at least one beam of ultrasounds is generated, focused and directed toward a bone region of a patient, such that the beam focus is matched with a specific point of the bone region. The ultrasound beam hits the bone region with an intensity and for a duration such to cause a hematoma and/or local inflammation and/or a series of micro-fractures in the target bone region, thereby activating a mechanism of osteogenesis.

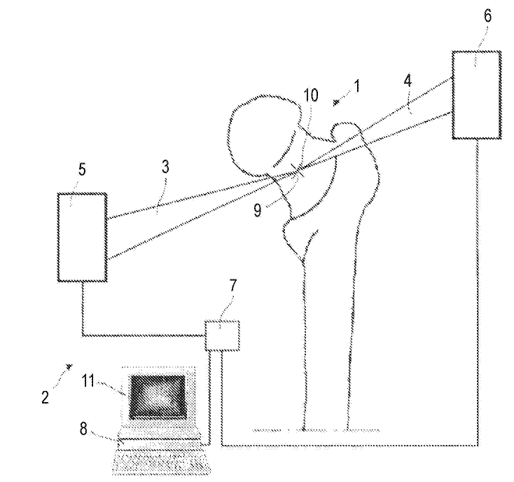

### FIELD OF THE INVENTION

The present invention concerns a method and equipment to induce osteogenesis in a bone region of a patient.

### BACKGROUND OF THE INVENTION

The problem of fractures in osteoporotic subjects, especially in the femur, is of major significance in the social, health and human fields.

In the last thirty years, an exponential growth of clinical cases and a consequential increase in social costs has been observed due to the aging process of the world population and to changing lifestyles.

The phenomenon, once especially concerning the Western and the North American countries (because of the common economic welfare), is now spreading through the Oriental countries.

Moreover, femural fractures, once regarding in the vast majority the female population, are nowadays widely affecting the male population.

Prevention attempts in the medical field (antiosteoporotics and recalcification substances) have not succeeded in bettering statistical data of femural fractures.

Trying to reduce the problem range, a “support” system has been suggested, to strengthen the bone zone most subjected to spontaneous femural fracture; the system consists in the preventive introduction of a metal “nail”, after a bone densitometry estimation of the potential femural fracture.

The named method has not been followed, for it is extremely invasive and it involves medical and legal risks.

### SUMMARY OF THE INVENTION

The aim of the present invention is to supply a method and equipment to induce osteogenesis in a bone region of a patient, with particular reference to the neck of the femur, this zone being the mostly often affected by the spontaneous fracture due to osteoporosis (Ward Triangle).

The present invention relates to a method for inducing osteogenesis in a bone region of a patient, which includes the steps of producing one or more ultrasound beams; focusing the one or more ultrasound beams; and directing the one or more ultrasound beams to a predetermined target area of the bone region. The power and duration of the one or more ultrasound beams are selected by a clinician to cause, in the bone region, a hematoma and/or a local inflammation and/or a series of micro-fractures, next to which an osteogenetic mechanism will be activated.

The present invention also relates to equipment for inducing osteogenesis in a bone region of a patient.

### BRIEF DESCRIPTION OF THE DRAWINGS

The present invention will be described with reference to the enclosed drawing, which depicts and schematizes an exemplary embodiment of the invention.

### DETAILED DESCRIPTION OF THE DRAWINGS

Number 1 in the FIGURE indicates a bone portion of a patient to be subjected to treatment. More particularly, the FIGURE depicts the upper portion of a femur (neck of the femur).

Number 2 indicates a piece of equipment for the emission of at least two focalized beams 3 and 4 of ultrasounds, which are emitted and focalized, in a way itself known, by respective emitter-elements 5 and 6 which are reciprocally connected in a not-shown way.

The emitter elements 5 and 6 are able to move in any direction due to the operation of a power element 7, of a type known in the art, which is activated with a mode also known in the art and later on clarified, by an operator working on a computerized unit 8.

Obviously, as an alternative to the movement of the emitter-elements 5 and 6, the power-elements 7 could vary the position of any mechanical or optical devices configured to modify the positions of the focuses of the focalized ultrasounds beams 3 and 4.

Having acquired and demonstrated by recent studies that the regenerative process of the bone consists in two types of osteogenesis, the first one being static and the second one dynamic, the use of the equipment 2 is estimated in accordance with the present invention to induce local micro-injuries in a bone region of a patient, particularly in the neck of the femur 1 in the zone statistically most affected by spontaneous femural fractures due to osteoporosis (Ward triangle), considering that it has been demonstrated that the regenerating process of the bone do substantially correspond to the event-sequence of the normal bone histogenesis.

The osteogenetic process follows two schemes: a static osteogenesis and a dynamic osteogenesis.

Around and in the bone injury, after the hematoma phase and the inflammatory process, the presence of a connective tissue rich of blood vessels can be noticed. Afterwards, in the area among the vessels, stable osteoblast cords are to be noticed, which do not move but change into osteocytes in the same place where they differentiate and originate a bone trabecula, thereby providing static osteogenesis.

In the meantime, the typical floating osteoblasts form strata on the surface of the trabecula shaped by static osteogenesis and they thicken them, thereby providing dynamic osteogenesis.

It is to be noted that in both the osteogenesis types are involved different signals and factors both in normal and in pathological conditions.

The static osteogenesis depends on inductive factors, the dynamic osteogenesis mostly depends on mechanical stimuli.

Using the equipment 2 to “hit” some internal portions of the neck of the femur 1 (e.g. portion 9) with focused ultrasounds beams 3 and 4, converging towards the internal portions themselves next to their focuses, it is possible to provoke a calculated series of “micro-fractures” in the neck of the femur zone 1, next to which the osteogenetic mechanism of both of the above described types will be activated.

The focused ultrasounds beams 3 and 4, when reaching and superposing the precise points chosen by the operator, produce a resonance effect together with a heat effect which induce hematoma and local inflammation.

This process is to be preferably checked by the computerized unit 8, which very precisely regulates the power of the focused beams 3 and 4 avoiding the creation of zones of lower resistance, which could lead to spontaneous fractures of the neck of the femur.

When exposing the osteoporotic femur to successive sessions of ultrasound “firing”, the affected zone will regain its strong ultrastructure of bearing bone.

The bone region 1 is preferably displayed by radioscopy, so that the operator who activates the equipment 2 can clearly visualize the bone portions that are subjected to the effect of the ultrasound focused beams 3 and 4.

Alternatively, the bone region 1 could be displayed, as a further example, using equipment for magnetic resonance imaging.

Preferably, the display of the radioscopic system or of the magnetic resonance imaging equipment is, in a way known in the art, fed into the computerized unit 8, supplied with references, so that the computerized unit 8 itself will “know” the coordinates of every displayed point and is able to show the result of the radioscopy or of the magnetic resonance imaging by a video terminal 11; it will be similarly advantageous that the position of the coinciding focus of the ultrasounds focused beams 3 and 4 is clearly identified and visualized in the same video terminal 11, second after second.

The coordinates of the points and the zones to be hit by the beams 3 and 4 being known, and the coordinates of the point 10 where the focused beams 3 and 4 intersect being known too, for they are dimensional features of the equipment 2, and having a display of the points and the zones in the video terminal 11, it will be easy for the operator who is working on a control element such as a joystick (not illustrated) connected to the computerized unit 8 to fix the movement of the emitter elements 5 and 6 through the power element 7 in order to identify the point 10 matching the points of the neck of the femur 1, where the beams 3 and 4 must operate.

With the present method and equipment, it will be possible to give origin to the above-mentioned calculated series of micro-fractures in the neck of the femur 1, producing ultrasound beams 3 and 4 having properly determined power, and the above-stated osteogenetic mechanism will be activated next to the micro-fractures.

When wished, the equipment 2 could provide for the use of only one of the beam 3, 4, or of any number of ultrasound focused beams, more than two, converging to a point.

What is claimed is:

1. A method of inducing osteogenesis in a bone region of a patient comprising:
producing at least one beam of ultrasounds;
focusing the at least one beam of ultrasounds;
directing the at least one beam of ultrasounds to the bone region of the patient, causing the focus of the at least one beam of ultrasounds to coincide with a predetermined point of the bone region;
wherein power and duration of the at least one beam of ultrasounds on the bone region are selected to cause, in an area of the bone region, a hematoma and/or a local inflammation and/or a series of micro-fractures, next to which an osteogenetic mechanism will be activated.

2. The method according to claim 1, wherein the at least one beam comprises two beams focused to converge into the predetermined point of the bone region.

3. The method according to claim 1, further comprising subjecting the bone region to a radioscopy, the result of the radioscopy being displayed by a video terminal connected to a computerized unit supplied with references, such that the computerized unit determines coordinates of every point visualized by the radioscopy, and such that a position of the focus of the at least one beam of ultrasounds is clearly identified and displayed in the video terminal over predetermined time intervals.

4. The method according to claim 1, further comprising exploring the bone region with magnetic resonance imaging, a result of the exploration being displayed by a video terminal connected to a computerized unit supplied with references, such that the computerized unit determines coordinates of every displayed point, and such that a position of the focus of the at least one beam of ultrasounds is clearly identified and visualized in the video terminal over predetermined time intervals.

5. The according to claim 1, further comprising the step of enabling an operator to modify a position of the focus of the at least one beam of ultrasounds to match the focus with the predetermined point of the bone region.

6. The method according to claim 5, wherein the operator is enabled to modify a direction of spreading of the at least one beam of ultrasounds by operating a joystick connected to a computerized unit.

7. Equipment to induce osteogenesis in a bone region of a patient, comprising:
an emitter configured to generate at least one focused beam of ultrasounds and to direct the at least one focused beam of ultrasounds to the bone region, the emitter providing the focus of the at least one beam at a predetermined point of the bone region,
wherein power and duration of the at least one focused beam on the bone region are selected to cause a hematoma and/or a local inflammation and/or a series of micro-fractures, next to which an osteogenetic mechanism will be activated.

8. The equipment according to claim 7, further comprising a power system configured to modify a direction of spreading of the at least one focused beam of ultrasounds.

9. The equipment according to claim 7, wherein there are two focused beams of ultrasounds.

10. The equipment according to claim 7 further comprising a computerized unit to which a video terminal is connected, wherein the bone region is subjected to radioscopy and the result of the radioscopy is displayed by the video terminal, together with references, such that the computerized unit identifies coordinates of every point displayed through the radioscopy, and such that a position of the focus of the at least one focused beam of ultrasounds is clearly identified and displayed on the video terminal over predetermined time intervals.

11. The equipment according to claim 7, further comprising a computerized unit to which a video terminal is connected, wherein the bone region is explored by magnetic resonance imaging, such that a result of the exploration is displayed by the video terminal together with references, such that the computerized unit determines coordinates of every displayed point, and such that a position of the focus of the at least one focused beam of ultrasounds is clearly identified and displayed on the video terminal.

12. The equipment according to the claim 7, further comprising a control unit configured to modify, in use, a position of the focus of the at least one focused beam of ultrasounds and to direct the at least one focused beam of ultrasounds over the predetermined point of the bone region.

13. The equipment according to the claim 12, wherein the control unit comprises a joystick movable by an operator and connected to a computerized unit operatively coupled to imaging equipment and wherein the imaging equipment provides one or more images of the bone region on a video terminal together with references, such that the computerized unit determines coordinates of every displayed point, and such that a position of the focus of the at least one focused beam of ultrasounds is clearly identified and displayed on the video terminal.

14. The method according to claim 1, wherein the bone region is a neck of the femur.

15. The equipment according to claim 7, wherein the bone region is the neck of the femur.
